(19) United States
(12) Patent Application Publication
(10) Pub. No.: US 2020/0360641 A1
(43) Pub. Date: Nov. 19, 2020

(54) MODULAR PULMONARY TREATMENT SYSTEM

(71) Applicant: AEON RESEARCH AND TECHNOLOGY, INC., Hewlett, NY (US)

(72) Inventors: Sunil Kumar Dhuper, Old Westbury, NY (US); Greg Marler, Rockford, IL (US)

(21) Appl. No.: 16/874,048

(22) Filed: May 14, 2020

Related U.S. Application Data
(63) Continuation of application No. 14/958,706, filed on Dec. 3, 2015, now abandoned.
(60) Provisional application No. 62/088,139, filed on Dec. 5, 2014.
(60) Provisional application No. 62/143,506, filed on Apr. 6, 2015.

(51) Int. Cl. A61M 16/08; A61M 16/20; A61M 11/06; A61M 15/00; A61M 16/06; A61M 16/14

(52) U.S. Cl. CPC A61M 16/0816; A61M 16/208; A61M 16/0833; A61M 11/06; A61M 2202/0208; A61M 16/205; A61M 15/009; A61M 16/06; A61M 16/14; A61M 16/204

(57) ABSTRACT

A modular pulmonary treatment system is provided and includes a patient interface device defined by a body which defines a hollow interior. The system also includes a first inhalation valve associated with the body and located along an inhalation flow path for providing selective fluid communication with the hollow interior of the body; and at least one exhalation valve assembly detachable coupled to the body for discharging exhaled gas from the hollow interior to atmosphere. The at least one exhalation valve assembly comprises: (a) an exhalation valve cartridge including a housing having a perforated bottom wall with a post extending outwardly therefrom; (b) a valve member configured to seat against the perforated bottom wall and including an opening through which the post extends; and (c) a valve retainer for placement over the post, whereby the valve member is securely held in place against the perforated bottom wall.

### CROSS REFERENCE TO RELATED APPLICATION

The present application is a continuation of U.S. patent application Ser. No. 14/958,706, filed Dec. 3, 2015, which claims the benefit of and priority to U.S. provisional patent application Nos. 62/088,139, filed Dec. 5, 2014, and 62/143,506, filed Apr. 6, 2015, each of which is hereby expressly incorporated by reference in its entirety.

### TECHNICAL FIELD

The present invention relates to pulmonary treatment equipment and more particularly, relates to a modular pulmonary treatment system that includes a number of interchangeable parts that allow the system to have a number of different operating modes including but not limited to delivery of a gas to a patient; delivery of an aerosolized medication (drug) to a patient; and a combination thereof.

### BACKGROUND

Respiratory care devices are commonly used as a means to deliver gases and medication in an aerosolized form to a patient. Aerosolized medication is typically used to treat patients with respiratory conditions, such as reactive airways disease, asthma, bronchitis, emphysema, or chronic obstructive pulmonary disease (COPD), bronchiectasis, cystic fibrosis, etc.

It is generally accepted that effective administration of aerosolized medication depends on the delivery system and its position in relation to the patient. Aerosol particle deposition is influenced by particle size, ventilatory pattern, and airway architecture, and effective medication response is influenced by the dose of the medication used.

An aerosol delivery system includes three principal elements, namely a generator, a power source, and an interface. Generators include small volume nebulizers (SVN), large volume nebulizers (LVN), metered dose inhalers (MDI), and dry powder inhalers (DPI). The power source is the mechanism by which the generator operates or is actuated and includes compressed gas for SVN and LVN and self-contained propellants for MDI. The interface is the conduit between the generator and the patient and includes spacer devices/accessory devices with mouthpieces or face masks. Depending on the patient's age (ability) and coordination, various interfaces are used in conjunction with SVN and MDI in order to optimize drug delivery.

The three primary means for delivering aerosolized medication to treat a medical condition is an MDI, a DPI, or a nebulizer. MDI medication (drug) canisters are typically sold by manufacturers with a boot that includes a nozzle, an actuator, and a mouthpiece. Patients can self-administer the MDI medication using the boot alone but the majority of patients have difficulty synchronizing the actuation of the MDI canister with inhalation causing oropharyngeal drug deposition, decreased drug delivery and therefore effectiveness, and causes other adverse effects.

A dry powder inhaler (DPI) is a device that delivers medication to the lungs in the form of a dry powder. DPIs are an alternative to the aerosol based inhalers commonly called metered-dose inhaler (or MDI). The DPIs may require some procedure to allow a measured dose of powder to be ready for the patient to take. The medication is commonly held either in a capsule for manual loading or a proprietary form from inside the inhaler. Once loaded or actuated, the operator puts the mouthpiece of the inhaler into their mouth and takes a deep inhalation, holding their breath for 5-10 seconds. There are a variety of such devices. The dose that can be delivered is typically less than a few tens of milligrams in a single breath since larger powder doses may lead to provocation of cough. Most DPIs rely on the force of patient inhalation to entrain powder from the device and subsequently break-up the powder into particles that are small enough to reach the lungs. For this reason, insufficient patient inhalation flow rates may lead to reduced dose delivery and incomplete deaggregation of the powder, leading to unsatisfactory device performance. Thus, most DPIs have a minimum inspiratory effort that is needed for proper use and it is for this reason that such DPIs are normally used only in older children and adults.

Small volume nebulizers (SVN) and large volume nebulizers (LVN) have been used to overcome difficulties encountered with MDI and DPI during acute exacerbation of obstructive airways disease but even these devices are fraught with problems especially significant waste of medication and not adequately reaching the target airways.

Problems with prior art devices include that the devices are inefficient and significantly waste medication, they provide a non-uniform concentration of delivered medication, they are expensive, and they are difficult to use. In addition, multiple pieces of equipment are needed to treat a plurality of different conditions.

The modular pulmonary treatment system of the present invention overcomes these deficiencies and provides a system that includes a number of interchangeable parts that allow the system to have a number of different operating modes including but not limited to delivery of a gas to a patient; delivery of an aerosolized medication (drug) to a patient; and a combination thereof.

### SUMMARY

In one aspect of the present invention, a modular pulmonary treatment system is provided and includes a patient interface device defined by a body which defines a hollow interior. The system also includes a first inhalation valve associated with the body and located along an inhalation flow path for providing selective fluid communication with the hollow interior of the body; and at least one exhalation valve assembly detachable coupled to the body for discharging exhaled gas from the hollow interior to atmosphere. The at least one exhalation valve assembly comprises: (a) an exhalation valve cartridge including a housing having a perforated bottom wall with a post extending outwardly therefrom; (b) a valve member configured to seat against the perforated bottom wall and including an opening through which the post extends; and (c) a valve retainer for placement over the post, whereby the valve member is securely held in place against the perforated bottom wall.

The body includes at least one exhalation opening with the exhalation valve cartridge being secured to the body such that the exhalation valve cartridge covers the exhalation opening and the exhalation valve cartridge is detachable as a whole unit from the body.

### BRIEF DESCRIPTION OF THE DRAWING FIGURES

FIG. 1 is an exploded perspective view of a patient interface device according to a first exemplary embodiment and for use with a breathing system;

FIG. 2 is a perspective view of the patient interface device in an assembled condition;

FIG. 3A is an exploded perspective view of a valve assembly for use with the patient interface device;

FIG. 3B is a perspective view of the valve assembly in a fully assembled condition;

FIG. 4A is an exploded view of a conduit member that is part of the patient interface device;

FIG. 4B is a perspective view of the conduit member in a fully assembled condition;

FIG. 5 is an exploded perspective view of a patient interface device according to a second exemplary embodiment and for use with a breathing system;

FIG. 6 is a perspective view of the patient interface device in an assembled condition;

FIG. 7 is an exploded perspective view of a patient interface device according to a third exemplary embodiment and for use with a breathing system;

FIG. 8A is an exploded view of a conduit member that is part of the patient interface device;

FIG. 8B is a perspective view of the conduit member in a fully assembled condition;

FIG. 9 is a perspective view of the patient interface device in an assembled condition;

FIG. 10 is an exploded perspective view of a patient interface device according to a fourth exemplary embodiment and for use with a breathing system;

FIG. 11 is an exploded view of a conduit member that is part of the patient interface device;

FIG. 12 is a perspective view of the conduit member in a fully assembled condition;

FIG. 13 is a perspective view of the patient interface device in an assembled condition;

FIG. 14 is a left side perspective view of the patient interface device of one embodiment of the present invention;

FIG. 15 is a schematic showing the use of the patient interface device of FIG. 14 as part of a metered port system;

FIG. 16 is a perspective view of a patient interface device of one embodiment of the present invention;

FIG. 17 is a perspective view of a patient interface device of one embodiment of the present invention;

FIG. 18 is a perspective view of a patient interface device of one embodiment of the present invention;

FIG. 19 is a perspective view of a patient interface device of one embodiment of the present invention;

FIG. 20 is a perspective view of a patient interface device of one embodiment of the present invention;

FIG. 21 is a perspective view of a patient interface device of one embodiment of the present invention;

FIG. 22 is a perspective view of a patient interface device of one embodiment of the present invention;

FIG. 23 is a perspective view of a patient interface device of one embodiment of the present invention; and

FIG. 24 is a perspective view of a patient interface device of one embodiment of the present invention.

### DETAILED DESCRIPTION OF CERTAIN EMBODIMENTS

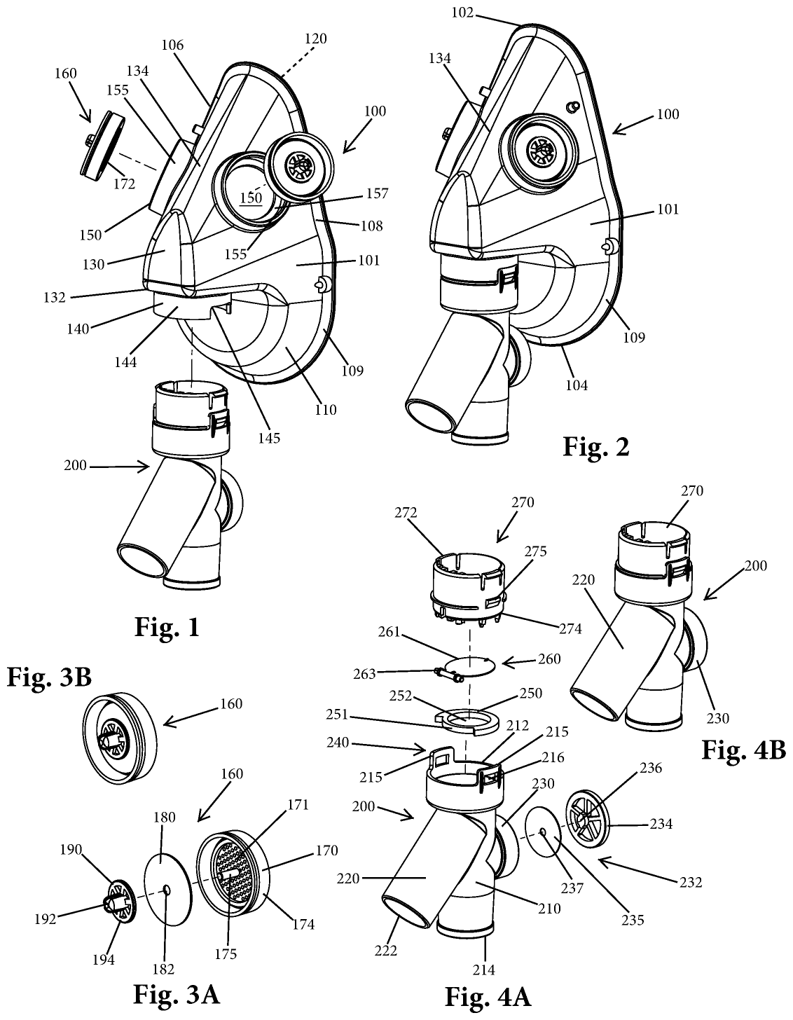

FIGS. 1-4B illustrate a patient interface device 100 according to a first embodiment. The patient interface device 100 is intended to deliver a gas to a patient as part of a breathing system such as the ones described in commonly assigned U.S. patent application Ser. No. 13/747,095, filed Jan. 22, 2013, which is hereby incorporated by reference in its entirety. The patient interface device 100 can be in the form of a face mask for application to a face of the patient. The patient interface device 100 includes a body 101 in the form of a face mask body that includes a first face 110 that represents an outer surface and an opposite second face 120 that represents an inner surface. The body 101 has a top 102, a bottom 104, a first side 106 and an opposite second side 108.

It will be appreciated that the body 101 can have any number of different structure and shapes and the face mask body 101 shown in the figures is merely exemplary and not limiting of the present invention. For example, the face mask body 101 can include a perimeter flange 109 or the like that is flexible for providing a seal to the patient's face.

The face mask body 101 includes a nose portion 130 that is defined by a generally planar underside 132 and a front angled portion 134. An angle is thus formed and defined between the planar underside 132 and the front angled portion 134. The face mask body 101 is a generally hollow structure and in particular, the second face 120 is open and defined an entrance into a hollow interior into which the patient's face is received.

The generally planar underside 132 includes a connector portion or structure 140. The connector portion 140 depends downwardly from the underside 132 and is configured to mate with and be coupled to another structure, in this case, a conduit member 200. The connector portion 140 includes a first coupling structure 142 that provides a means for coupling the face mask body 101 to the conduit member 200. The connector portion 140 can be in the form of a wall structure 144 (e.g., a circular shaped wall) that includes one or more notches 145 (or slots or openings). The first coupling structure 142 can be of the type that promotes a snap-fit connection between the face mask body 101 and the conduit member 200.

The generally planar underside 132 of the nose portion 130 includes an opening that is axially aligned with the hollow interior of the connector portion 140 so to provide fluid communication into the hollow interior of the mask body 101 through the hollow connector portion 140. As described herein, when the hollow conduit member 200 is mated to the connector portion 140, the hollow interior of the conduit member 200 is thus in fluid communication with the hollow interior of the face mask body 101.

The face mask body 101 also includes one or more outlets 150. The illustrated face mask body 101 includes two outlets 150 in the form of through holes formed through the face mask body 101. One outlet 150 can be formed on one side 106 of the face mask body 101 and the other outlet 150 can be formed on the other side 108 of the face mask body 101. In one embodiment, the outlets 150 are in the form of circular shapes holes formed in the face mask body 101. Around each outlet (hole) 150 is an upstanding wall (flange) 155 that extends around the hole 150. The upstanding wall 155 can thus be in the form of a circular shaped wall (flange). Inside the outlet 150, there is a landing/platform 157 formed between the inner surface of the upstanding wall 155 and the through hole 150. This landing/platform 157 is annular shaped.

The outlets 150 serve as exhalation ports and include an exhalation valve assembly 160. The exhalation valve assembly 160 includes a housing 170, a valve member 180, and a valve retainer 190. The housing 170 is configured to be received within the outlet 150 and more specifically, the housing 170 is received within and between the upstanding wall 155. When the upstanding wall 155 has a circular shape, the housing 170 has a complementary circular shape. The size of the housing 170 is selected so that when the housing 170 is received within the outlet 150, a bottom surface 172 of a bottom wall 171 of the housing 170 sits against and is supported by the landing/platform 157. A side wall 174 of the housing 170 seats against the inner surface of the upstanding wall 155. The side wall 174 extends upwardly from the bottom wall 171 and is formed along the perimeter of the bottom wall 171.

As shown in the figures, the bottom wall 171 is configured to allow fluid (air) to flow therethrough and in particular, the bottom wall 171 can be a mesh structure or otherwise include one or more openings to allow the fluid (exhausted gas) to pass therethrough.

The housing 170 also includes an upstanding post 175 that is fixedly attached to (e.g., integrally formed with) the bottom wall 171. In the illustrated embodiment, the post 175 has a circular shape and is centrally formed on the bottom wall 171. The post 175 can thus be in the form of a cylindrical shaped post that is centrally located.

The valve member 180 is a flexible structure that serves to selectively close off the valve assembly 180 under select conditions. The illustrated valve member 180 has a circular shape with a center opening 182. The center opening 182 is configured to receive the post 175, thereby coupling the valve member 180 to the housing 170. When the valve member 180 seats flush against the bottom wall 171, the valve assembly 160 is in a closed position and fluid cannot flow therethrough. When the valve member 180 unseats relative to the bottom wall 171, the valve assembly 160 is in an open position and fluid flows and in this case, exhaled air is vented.

The valve retainer 190 serves to hold the valve member 180 in place on the post 175, while permitting normal operation and movement of the valve member 180 during the patient's inhalation and exhalation. The valve retainer 190 is a thimble-like structure that has a hollow center boss 192 and a flange 194 extending radially outward therefrom. The flange 194 can include openings as shown. The boss 192 has a cylindrical shape and is configured to receive the post 175. When assembled, the valve member 180 is disposed between the valve retainer 190 and the housing 170. Any number of techniques can be used to couple the valve retainer 190 to housing 170 including a mechanical fit (friction, snap-fit, etc.).

As shown in FIG. 1, a top of the valve retainer 190 extends above a top edge of the side wall 174 when the valve retainer 190 is mated to the post 175.

Since the exhalation valve is in the form of the valve assembly 160, the exhalation valves are freely insertable and removable from the mask body 101 since they are in cartridge form.

The valve assembly 160 comprises a one way valve and as mentioned previously, the valve assembly 160 functions as an exhalation valve that serves to discharge exhausted air from the patient.

The housing 170 is configured to be received within the outlet 150 and more specifically, the housing 170 is received within and between the upstanding wall 155. A friction fit can be used to couple the housing 170 to the outlet 150. The valve assembly 160 can thus be thought of as being a cartridge like assembly that is inserted to the outlet 150. Since the valve assembly is in cartridge form, if for whatever reason one of the valve assemblies 160 needs to be changed and/or repaired, the assembly 160 can be simply removed from the outlet 150 to accomplish such task. Any number of other coupling techniques can be used such as a releasable snap fit between the assembly 160 and the outlet 150.

When the upstanding wall 155 has a circular shape, the housing 170 has a complementary circular shape. The size of the housing 170 is selected so that when the housing 170 is received within the outlet 150, a bottom surface 172 of a bottom wall 171 of the housing 170 sits against and is supported by the landing/platform 157. A side wall 174 of the housing 170 seats against the inner surface of the upstanding wall 155.

The conduit member 200 is best shown in FIGS. 4A and 4B. The conduit member 200 includes a main conduit body 210 that includes a first end (top) 212 and an opposing second end (bottom) 214. The main conduit body 210 is a hollow structure that also includes a side port 220 that is a circular shaped tubular structure that extends outwardly from the main conduit body 210. The side port 220 terminates in a free distal end 222. The inside of the side port 220 is in fluid communication with the hollow interior of the main conduit body 210. As shown, the side port 220 is formed at an angle relative to the main conduit body 210. The distal end 222 of the side port 220 can be above the second end 214 of the main conduit body 210. As discussed herein, the side port 220 represents a conduit through which fluid flows and thus, the side port 220 can allow fluid to flow into the conduit member 200 and to the face mask and vice versa. As shown, in use, the side port 220 faces forward in that it projects forwardly of the face mask.

As discussed, the conduit member 200 is a drug delivery component and the side port 220 is intended for delivery of medication (drug).

The main conduit body 210 also includes another side port 230 which is located opposite the side port 220 and therefore, in use, the side port 230 represents a rear port. The side port 230 can be in the form of a circular shaped port defined by a side wall 232 (circular shape). As with the side port 220, the side port 230 defines a fluid passage into the hollow interior of the main conduit body 210. The side port 230 can be formed generally perpendicular to the main conduit body 210.

The first end 212 of the main conduit body 210 has a coupling structure 240 for attaching the main conduit body 210 to another structure. The coupling structure 240 can be configured to snap-fittingly attach the first end 212 to the other structure. In particular, the first end 212 can be in the form of a pair of upstanding tabs 215. The tabs 215 have central openings (e.g., rectangular shaped openings) 216 formed therein. The tabs 215, as illustrated, can be formed about 180 degrees apart from one another. The tabs 215 extend above the first end 212.

The coupling structure 240 can include an inhalation assembly and more specifically, the coupling structure 240 can include a valve seat 250, a valve member 260, and a top retaining member 270. The valve seat 250 can be an annular shaped structure with a central opening 252. The valve seat 250 can have a planar top surface on which the valve member 260 seats (rests). A notch 251 can be formed in the valve seat 250 to allow for movement of the valve member 260.

The valve seat 250 is disposed within the main conduit body 210 proximate the first end 212. Any number of different techniques, including a mechanical fit or coupling, can be used.

The valve member 260 comprises a flapper valve (swing valve) defined by a circular shaped body 261 and a coupling member 263 in the form of an axle or pin that has two free ends. The axle 263 is designed to allow the valve member 260 to rotate thereabout to allow the valve member 260 to rotate between an open position and a closed position. The two free ends of the axle 263 can be received in complementary coupling structures (such as clamps).

The top retaining member 270 is in the form of a tubular structure (e.g., circular shaped) that has a first end 272 and an opposing second end 274. The second end 272 can include the complementary structures for attaching to and retaining the axle 263. An outer surface of the top retaining member 270 includes retaining projections 275 that are designed to mate with the upstanding tabs 215 for coupling the top retaining member 270 to the first end 212 of the main conduit body 210. The projections 275 are received within the openings 216 for releasably retaining the top retaining member 270 to the main conduit body 210. The top retaining member 270 thus acts to lock the inhalation valve assembly in place within the main conduit body 210 proximate the first end 212.

The main conduit body 210 serves to provide an entrance for more or more gases including air as a result of the main conduit body 210 having an open second end 214 (to which a gas source can be connected) and the side port 220 (to which a gas source can be connected). The gas that flows into second end 214 and side port 220 can be different or the same. For example, one gas can be oxygen, while the other gas is a supplemental gas which can be different than oxygen or can be oxygen. The gas sources can be connected to these ports using conventional techniques, such as tubing and the like, which connect from the gas source (e.g., a tank) to the main conduit body 210.

The side port 230 is designed to act as an inhalation valve that opens under select conditions (e.g., conditions in which additional air is needed to be delivered to the patient). As a result, the side port 230 contains an inhalation valve assembly 232. The inhalation valve assembly 232 includes a valve retainer 234 and a valve member 235. The valve assembly 232 is configured to act as a one-way valve assembly in that the valve member 235 opens only under inhalation conditions. The valve retainer 234 can be a circular structure with a spoked construction with a center protrusion 236. The valve member 235 can be in the form of a circular shaped flexible valve with a center opening 237. When assembled, the peripheral edge of the valve member 235 seats on spoked construction of the retainer 234 and the center protrusion 236 is received within the center opening 237 to couple the valve member 235 to the valve retainer 234. The valve member 235 and valve retainer 234 are disposed within the side port 230. When certain inhalation conditions exist, the valve member 235 will lift away from the valve retainer 234, thereby opening up air flow into the hollow interior of the main conduit body 210.

The face mask body 101 can be formed of any number of different materials including but not limited to polymeric materials.

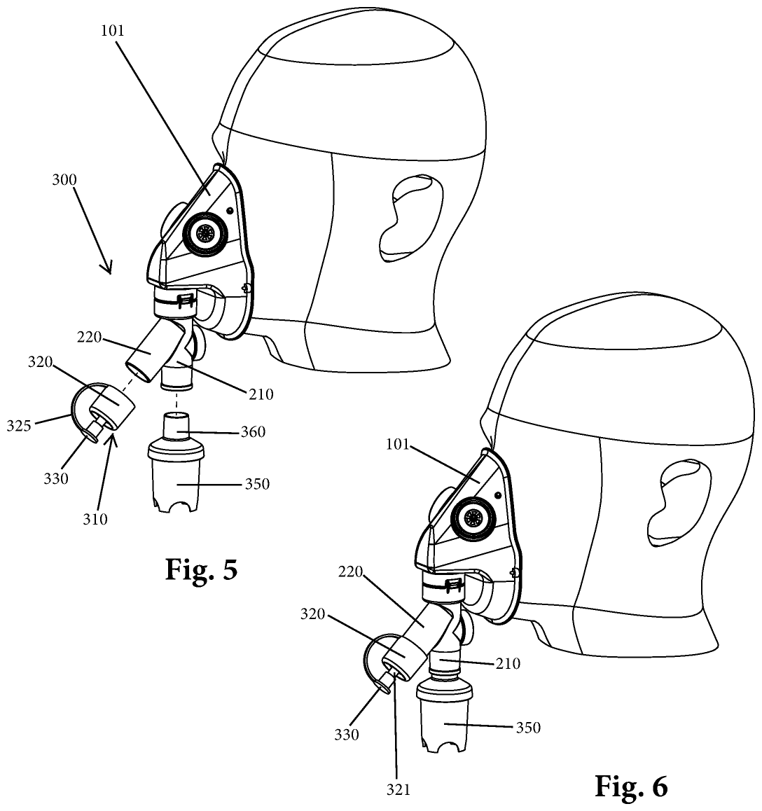

Now referring to FIGS. 5 and 6, a patient interface device 300 is shown and is similar to the patient interface device 100 and therefore, like elements are numbered alike. The patient interface device 300 includes the face mask body 101 and the conduit member 200. The side port 220 of the main conduit body 210 is connected to a cap 310 that is configured to sealingly and selectively close off the side port 220. The cap 310 includes a main cap body 320 that can be inserted into and/or mate to the end 222 of the side port 220. The cap 310 has a plug 330 that is tethered to the main cap body 320 with flexible tether 325. The plug 330 sealingly closes off the main cap body 320 when it is inserted therein. When the plug 330 is removed and detached from the main cap body 320, the main cap body 320 is open and a fluid conduit can be inserted therein for supplying a fluid to the side port 220 of the conduit member 200. For example, if it is desired to provide additional gas to the patient, a gas flow can be fluidly connected to the side port 220. For example, a gas tube with a connector can be mated to the main cap body 320 to provide gas flow to the main conduit body 210 and the face mask body 101.

More specifically, the main cap body 320 can include a gas connector (nipple) 321 to which a fluid conduit (tube) can be connected for delivering a fluid (gas) to the side port 220 and the patient interface device 100. A friction fit can be provided between the tube and the nipple 321 once the plug 330 is removed from the nipple 321. The plug 330 is thus configured to mate to and close off the nipple 321. The cap body 320 can be frictionally fit to the side port 220 to provide a fluid seal.

The patient interface device 300 also includes a nebulizer 350. As is known, a nebulizer 350 is a drug delivery device that is used to administer medication in the form of a mist inhaled into the lungs. The nebulizer 350 includes a connector 360 at one end that is configured to mate with the end 214 of the main conduit body 210. For example friction fit or other mechanical fit (e.g., snap-fit) can be used to detachably connect the nebulizer 350 to the main conduit body 210. Another portion of the nebulizer 350 is fluidly connected to a source of aerosolized medication.

When the nebulizer 350 is active, the plug 330 is either inserted into the main cap body 320 or fluid is flowing through the main conduit body 210.

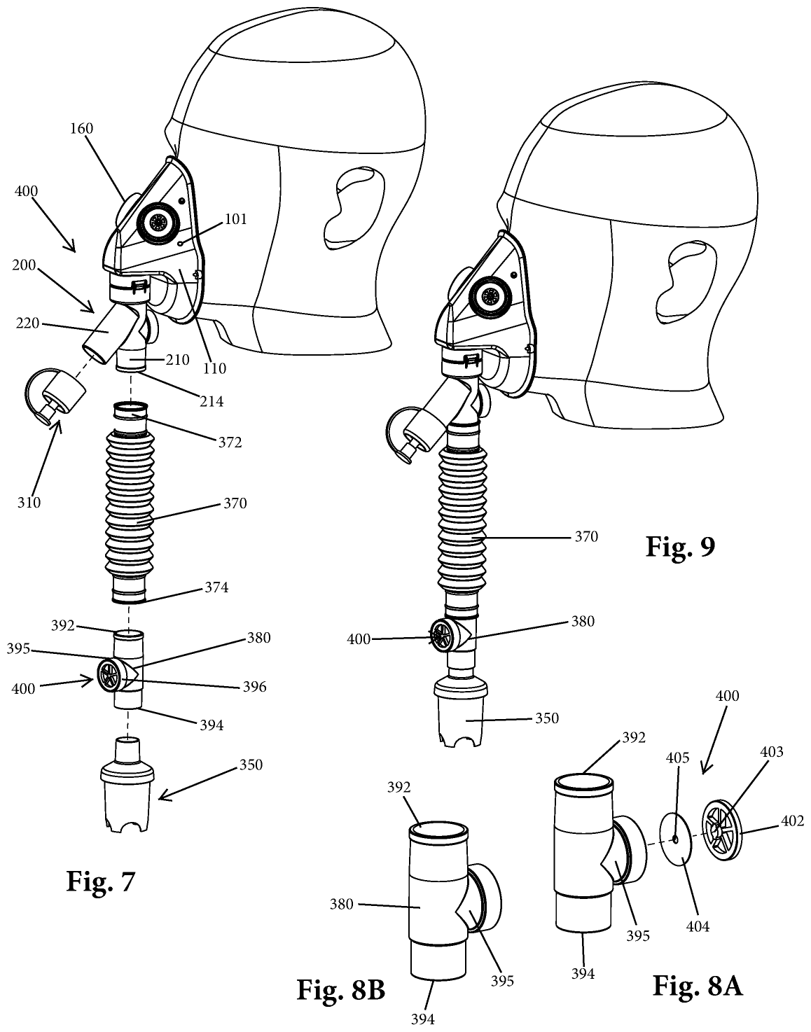

FIGS. 7-9 illustrate a patient interface device 400 is shown and is similar to the patient interface devices 100, 300 and therefore, like elements are numbered alike. The patient interface device 400 includes the face mask body 101 and the conduit member 200. The side port 220 of the main conduit body 210 is connected to the cap 310.

Instead of connecting the nebulizer 350 directly to the main conduit body 210, the nebulizer 350 is connected to the main conduit body 210 by a first connector 370 and a second connector 380. The first connector 370 has a first end 372 which attaches to end 214 of the main conduit body 210 and an opposite second end 374 which connects to the second connector 380. The second connector 380 attaches between the first connector 370 and the nebulizer 350.

The first connector 370 has an adjustable length in that it can be formed of corrugated tubing that has a bellows type construction. The adjustment of the length of the first connector 370 permits the length of the flow path of the aerosolized medication to likewise be adjusted (by either extending or retracting the first connector 370).

The second connector 380 includes a main body 390 that has a first end 392 and an opposing second end 394. Between the first and second ends 392, 394, the main body 390 has a side port 395. The side port 395 can be in the form of a circular shaped port defined by a side wall 396 (circular shape). The side port 395 defines a fluid passage into the hollow interior of the main conduit body 390. The side port 395 can be formed generally perpendicular to the main conduit body 390.

The side port 395 is designed to act as an inhalation valve that opens under select conditions (e.g., conditions in which additional air is needed to be delivered to the patient). As a result, the side port 395 contains an inhalation valve assembly 400. The inhalation valve assembly 400 includes a valve retainer 402 and a valve member 404. The valve assembly 400 is configured to act as a one-way valve assembly in that the valve member 404 opens only under inhalation conditions. The valve retainer 402 can be a circular structure with a spoked construction with a center protrusion 403. The valve member 404 can be in the form of a circular shaped flexible valve with a center opening 405. When assembled, the peripheral edge of the valve member 404 seats on spoked construction of the retainer 402 and the center protrusion 403 is received within the center opening 405 to couple the valve member 404 to the valve retainer 402. The valve member 404 and valve retainer 402 are disposed within the side port 390. When certain inhalation conditions exist, the valve member 404 will lift away from the valve retainer 402, thereby opening up air flow into the hollow interior of the main conduit body 390.

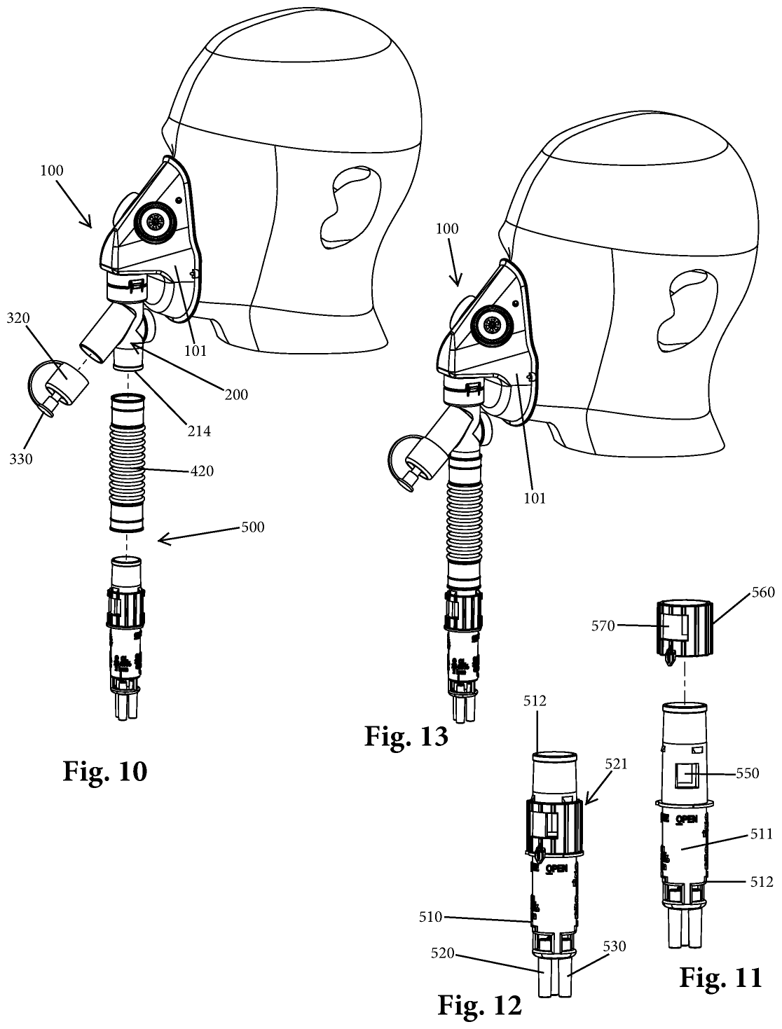

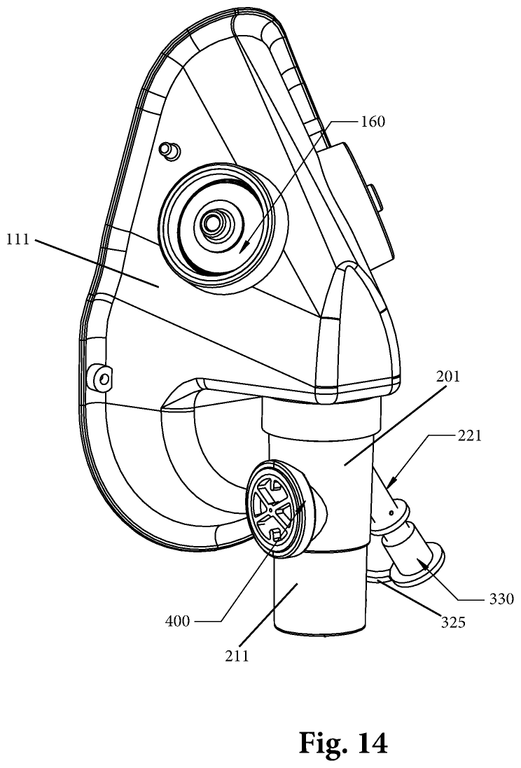

FIGS. 10-13 show another embodiment in accordance with the present invention and in particular, this embodiment includes a venturi assembly. One exemplary venturi assembly that can be used in the present invention is described in commonly owned U.S. patent application Ser. No. 13/748,305, filed Jan. 23, 2013, which is hereby incorporated by reference in its entirety.

FIG. 10 is an exploded perspective view of a venturi assembly 500 in accordance with another embodiment of the present invention. The assembly 500 is formed of a number of parts (components) that interact with one another to provide for controlled gas delivery to a patient. The assembly 500 is meant for use with a patient interface member (assembly) 100 that is designed to interact with the patient and in one exemplary embodiment, the interface member 100 is in the form of a mask assembly. It will be appreciated that the illustrated interface member is merely exemplary in nature and any number of other types of interface members can be used for delivering gas to the patient.

The end 214 of conduit member 200 receives the gas from the venturi assembly 500. An elongated conduit member 420 is connected to the end 214 of conduit member 200 and to the venturi assembly 500 for delivering the gas from the venturi assembly 500 to the interface member 100. The elongated conduit member 420 can be in the form of an elongated tube which can be of a type which is expandable/retractable in that a length of the elongated conduit member 420 can be varied. Conventional methods of attachment can be used to attach the elongated conduit member 420 to both the interface member 100 and the venturi assembly 500 (e.g., conical fitting, frictional fit, snap, etc. . . . ).

The venturi assembly 500 can be formed of two main components or as one part, and when two parts are used, the parts consist of a multi-port venturi member 510 and a secondary gas entrainment valve member 521. The multi-port venturi member 510 has a first end 512 and an opposite second end 514. The multi-port venturi member 510 is a generally hollow body 511 that includes a main hollow space. In the illustrated embodiment, the body 511 has a cylindrical shape; however, it will be appreciated that the body can have any number of other shapes.

The body 511 also has an air entrainment window 512 formed therein below the main hollow space. The air entrainment window 512 is thus open to atmosphere and serves to allow air to flow into the hollow space and then flow ultimately to the patient (by means of the elongated conduit member 420 and the interface member 100).

The member 510 also includes at least one and preferably a plurality of gas port members 520, 530 that extend downwardly from the lower body section. The gas port members 520, 530 are configured to be individually connected to a gas source (such as an oxygen gas source). The gas port members 520, 530 are elongated hollow conduits that each allows a fluid, such as gas (oxygen), to enter at an exposed, free distal end 520, 530 and flow therethrough into the hollow space while flowing by the air entrainment window (which is designed to allow atmospheric gas (air) to be entrained by the gas flow through the gas port members 520, 530). Entrainment of air through the window 512 results due to the pressure drop created by the gas that flows through either of the gas port members 520, 530. The distal ends can be barbed ends to facilitate mating of the gas port members 520, 530 to conduits (tubing) that is connected to the same, single gas source or to multiple gas sources.

In another embodiment, the member 510 includes only a single gas port member.

It will be understood that at any one operating time, gas is flowing through only one of the gas port members 520, 530. As described below, the gas port members 520, 530 have different gas flow characteristics and therefore, depending upon the desired gas concentration that is chosen to be delivered to the patent, the user selects one of the gas port members 520, 530 to use. Once again, at any one point in time, only one of the gas port members 520, 530 is active in that gas is flowing therethrough.

The gas port members 520, 530 are constructed so as to provide a known gas flow rate. In particular, a top wall is formed across the tops of the gas port members 520, 530 and defines the ceiling of the gas port members 520, 530. An orifice (through hole) is formed in the top walls of the gas port members 520, 530, respectively. The shape and dimensions of the orifices define the gas flow rates of the gas port members 520, 530 and more particularly, by varying the shape and size of the orifices, the gas flow rate associated with the gas port member is likewise changed.

As a result, the gas port member 520 can have one associated gas flow rate, while the gas port member 530 has a different gas flow rate associated therewith. It will be appreciated that the system 500 can include a plurality of single or multi-port venturi members that can be grouped as a kit. This allows the user to select the venturi member that has the desired, chosen gas flow rate. The venturi members can be interchanged as part of the overall system 500 depending upon the precise application and desired gas concentration to be delivered to the patient.

The tops of the gas port members 520, 530 can be disposed within the air entrainment window. In other words, the height of the gas port members 520, 530 is such that the tops are disposed within the air entrainment window 512 and therefore, gas exiting the top of one of the gas port members 520, 530 is mixed with entrained air flowing into the air entrainment window 512.

The gas flow rates associated with the gas port members 520, 530 can be the same or the flow rates can be different. The respective orifices can have different sizes and therefore, different flow rates. It will be appreciated that the orifices thus serve to meter the gas from the gas source as it flows through the gas port members 520, 530 into the hollow space.

The member 510 also includes a secondary window (air entrainment window 550) that is formed in the hollow body. The window 550 can be in the form of two distinct, defined windows that are located opposite one another. The window 550 is located above window 512. An additional part 560 mates with the hollow body and is in the form of a rotatable sleeve 560 that has a window 570 which can in the form of two distinct, defined windows that are located opposite one another. The sleeve 560 is inserted over the hollow body and there can be a lip of the like that positions the sleeve 560 in a target position in which the sleeve 560 is in registration with the secondary window 550 and more particularly, the window 570 is in registration with the window 550. It will be appreciated that the degree that the secondary window 550 is open to atmosphere depends on the degree of registration between the windows 550, 570. By rotating the sleeve 560, the degree of registration can be changed, thereby allowing more or less air to be entrained into the system.

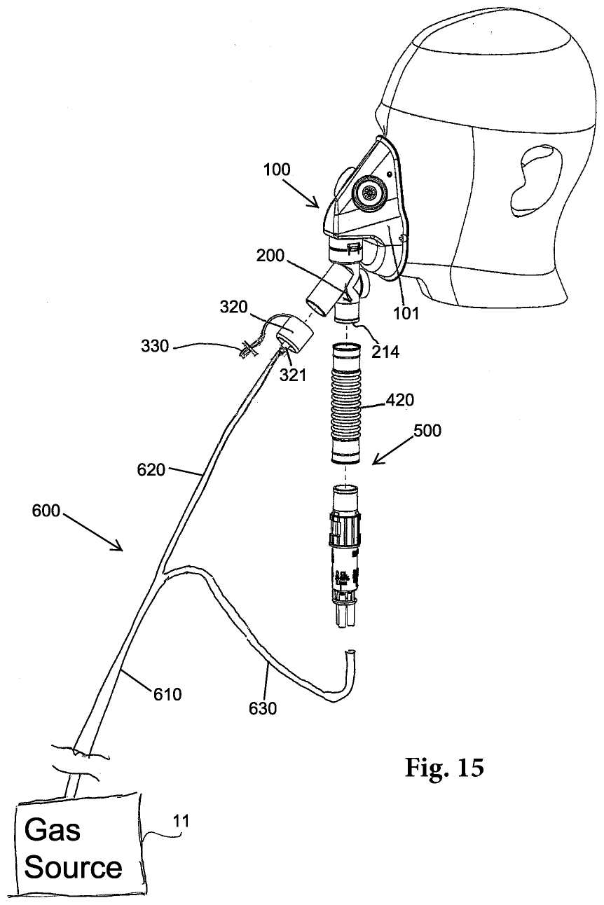

FIG. 14 illustrates a patient interface device 101 that is similar to the patient interface devices disclosed herein including the one in FIG. 5 and is constructed as a high precision aerosol/.venturi mask. FIG. 14 shows a conduit member 201 that is intended for gas delivery (e.g., oxygen delivery). The conduit member 201 is integrally connected to the mask 101 at the underside of the nose portion. The conduit member 201 has a main conduit 211 and a side port 221. In FIGS. 14 and 15, the side port 221 that extends outwardly from the main conduit body 210 is configured to mate directly to the fluid conduit (tube) that delivers a fluid (supplemental gas). In this embodiment, the tether 325 is connected to the side port 221 and the cap/plug 330 is configured to mate to and close off the open end of the side port 221. The side port 221 is formed at an angle, such as about a 45 degree angle. The patient interface device can have the features disclosed with respect to the other embodiments. More specifically, the patient interface device shown in FIG. 14 when combined with other components can provide the following features to the system: at least one inhalation valve, at least one exhalation valve 160 and optionally an emergency valve 400—each of which is described in detail with respect to the other embodiments.

Unlike the conduit member 200, the conduit member 201 does not include a flapper/swing valve assembly but instead only contains emergency inhalation valve assembly 400 along the main conduit 211. As shown in other figures, the conduit member 201 is intended to be disposed downstream of the conduit member 200 when the two are present in one system such that if there is a failure of the flapper valve or there is insufficient flow through the flapper valve, the emergency valve 405 will open and air flows to the patient.

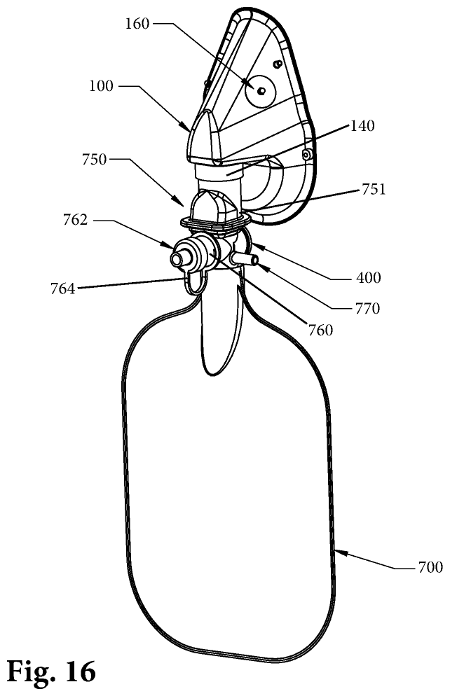

In accordance with the present invention, the system can include a means for controlling the composition of the supplemental gas delivered to the patient. For example, a gas source 11 (e.g., oxygen) can be fluidly connected to: (1) a metered port that is in fluid communication with the main conduit body 210 and (2) another device, such as venturi device 500, that controls the flow of the gas. In the illustrated embodiment shown in FIG. 16, a wye connector 600 is provided and includes a first main conduit 610 that is fluidly connected to the source 11. The wye connector 600 has a first branch conduit 620 and a second branch conduit 630. The wye connector 600 is thus formed such that the flow of gas from the source 11 is divided into the first and second branch conduits 620, 630 to deliver gas to the target locations.

The metered port can be in the form of the nipple 321 that is part of the cap body 320 (FIG. 15) or can be in the form of side port 220 as in FIG. 14. The metered port has a construction (e.g., diameter) that controllably allows only a predetermined quantity of gas into the main body 210. More specifically, the dimensions of the opening (flow path) formed in the metered port provide a given known flow rate of gas to the patient interface device.

The second branch conduit 630 is fluidly connected to the venturi device 500 for delivering gas thereto. In the embodiment of FIG. 15, the venturi device 500 is of a type that has a plurality of different inlet conduits that are configured to mate with the second branch conduit 630. In this design, only one of the inlet conduits of the venturi device 500 is fluidly connected to the second branch conduit 630 at any one time. Similar to the metered port, each of the inlet conduits of the venturi device has predetermined flow properties to control the flow of the gas to the patient through the patient interface device.

A flow control device can be disposed along the first branch conduit 620 to permit or restrict or prevent the flow of gas within the first branch conduit 620 to the patient interface device. It will be appreciated that in one operating scheme, gas flows through the metered port only when there is high demand for gas flow to the patient. In normal operating modes, gas can be routed such that it only flows within the second branch conduit 630 to the patient interface device.

The second branch conduit 630 can have no flow control devices to allow free flow of the gas to the patient interface device at all times.

As described herein, the venturi device, such as venturi device 500, can have a mechanism for altering the composition of gas that flows therethrough to the patient. For example, the secondary window of venturi device 500 can be moved between a number of different positions to control the level of air entrainment. By entraining air, the composition of the gas flowing into the venturi device 500 through the inlet can be varied.

It will be appreciated that the flow rates of the gas flowing through the venturi device 500 and through the metered port (example side port 220 in FIG. 14) can be the same or they can be different. Typically, the two are different and as mentioned herein, in many normal operating modes, the gas does not flow through the metered port but only flows from source 11 through the venturi device to the patient interface device.

In addition, in the embodiment shown in FIG. 14, the inhalation valve member 260 (a flapper valve/swing valve) can be eliminated and instead, the gas can freely flow into the patient interface device from the conduit 210. Exhaled gas does not travel down the conduit 210 but instead is exhaled through the one or more exhalation valves due to the flow rate of the gas in the conduit 210 in a direction toward to open interior of the patient interface device. In other words, the flow rate of gas in the conduit 210 is too great to permit exhaled gas to flow therein in an opposite direction from the gas being delivered to the patient.

It will also be understood that the construction of the first and second branch conduits can be different in that one can have a smaller diameter relative to the other. This is a way to control the flow rate of gas to the patient interface device.

The cap 330 thus can cap the metered port when it is not in use. In some operating modes, the metered port/side port for delivery of supplemental gas is not used and thus, it is sealingly capped.

FIG. 16 shows a system that combines a number of components described herein. The system of FIG. 16 can be thought of as being a 100% nonrebreather/aerosol drug delivery system (oxygen delivery only). The system includes the mask 100 and has a one-wave valve valve body connector 750 that incorporated a one-way inhalation valve. The valve body connector 750 can be similar to the conduit member 200 and can include a flapper or swing valve assembly 260 (inhalation valve) that opens upon inhalation. The flapper valve is generally located proximate the enlarged intermediate region 751. The valve body connector 750 is generally tubular in nature. An upper region of the valve body connector 750 is connected to the mask 100 as by mating with connector portion 140 that is integral to the mask. A lower region of the valve body connector 750 includes other connectors and ports as described herein. For example, a main port 760 can extend outwardly from the front of the valve body connector 750 (e.g., at a right angle). The main port acts as an aerosol/oxygen port that connects to oxygen or other gas or to a source of aerosolized medication. A cap 762 on a tether 764 can close off the main port 760. The valve body connector 750 also has a supplement air valve which can be in the form of valve assembly 400. A supplemental gas port 770 also is included and extends outwardly from the body of the valve body connector 750. Gas port 770 can be connected to a gas source, such as oxygen.

The valve body connector 750 can be considered a sub-assembly that is then subsequently attached to the mask 100 (as by connection to connector portion 140).

In the intermediate region 751 there is shown a pair of abutting flanges. In production, the top region of the component can be one part and the bottom region can be a separate second part. Each part has a flange and the two flanges are attached (e.g., ultrasonic welding) to complete the valve body connector 750. The initial separation of the parts allows for the insertion of the inhalation (flapper) valve.

Lastly, a distal end of the valve body connector 750 is open and can be attached to another component, such as a single reservoir bag 700.

The exhalation valve 160 can be a traditional exhalation valve or can have exhalation valve assembly 160.

The arrangement is FIG. 16 has the following features: 100% nonrebreather oxygen delivery; standard aerosol drug delivery; simultaneous oxygen and drug delivery capability (however, as shown, only oxygen is delivered); and a supplemental air valve.

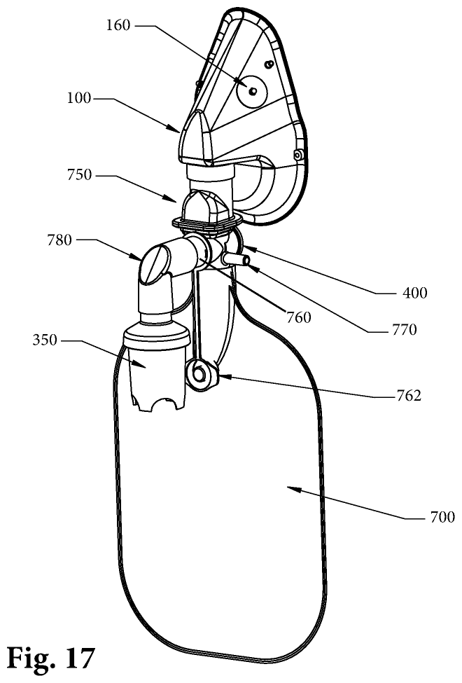

FIG. 17 shows another system that is similar to the one shown in FIG. 16 and therefore, the same components are numbered alike. This system is a 100% nonrebreather/aerosol drug delivery system (oxygen and drug delivery). In this arrangement, the main port 760 is connected to a connector 780 (e.g., elbow connector) which itself connects to the nebulizer 350.

The arrangement is FIG. 17 has the following features: 100% nonrebreather oxygen delivery; standard aerosol drug delivery; simultaneous oxygen and drug delivery; and a supplemental air valve.

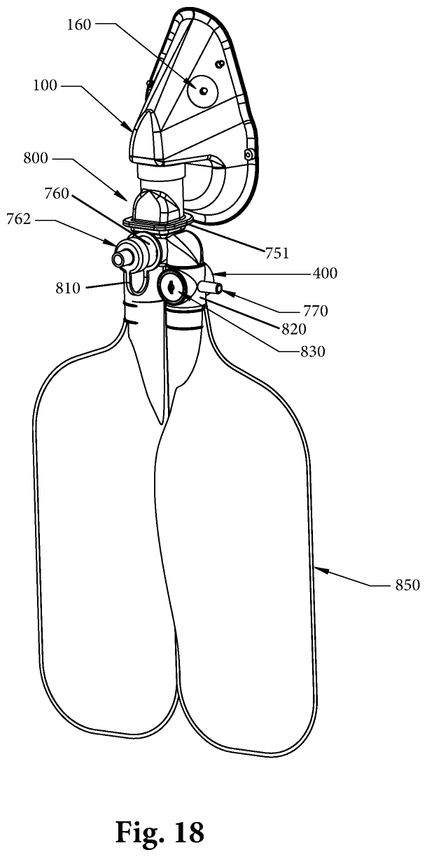

FIG. 18 shows another system that is similar to the previous ones and therefore, the same components are numbered alike. This system is a rescue 100% nonrebreather/high efficiency aerosol drug delivery system (oxygen delivery only). In this embodiment, a valve body connector 800 is used instead of valve body connector 750 due to the dual bag nature of this arrangement. The valve body connector 800 is similar to the valve body connector 750 and in fact the top regions of each can be the same. The component includes an inhalation valve assembly (such as valve assembly 260). The top region (tubular conduit) of the valve body connector 800 connects to the mask 100 (i.e., to the connector portion 140). The valve body connector 800 also includes the front main port 760 (as part of the bottom region). The bottom region also includes a pair of legs (tubular conduits) 810, 820. The first leg 810 connects to a complementary port/connector of a dual reservoir bag 850 and provides access to a first compartment of bag 850. The second leg 820 connects to a complementary port/connector of the dual reservoir bag 850 and provides access to a second compartment of bag 850. Along the second leg 820, the supplemental air valve (emergency inhalation valve) 400 and the oxygen port 770 are formed. In addition, a gas overflow valve 830 is provided in the leg 820 and serves to open when there is excessive pressure in the bag 850 (i.e., in the second compartment thereof). The valve 830 serves to vent excess stored gas to atmosphere to preserve the integrity of the bag 850.

The main port 760 is closed off with cap 762.

An open distal end of the second leg 820 connects to the bag 850.

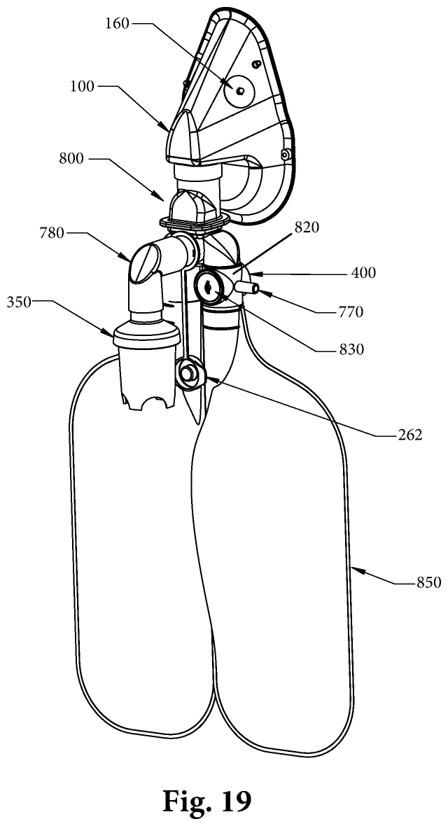

FIG. 19 shows a system similar to that shown in FIG. 18 with the exception that the main port 760 receives connector (elbow connector) 780. Nebulizer 350 is attached to the connector 780 and cap 762 is left off.

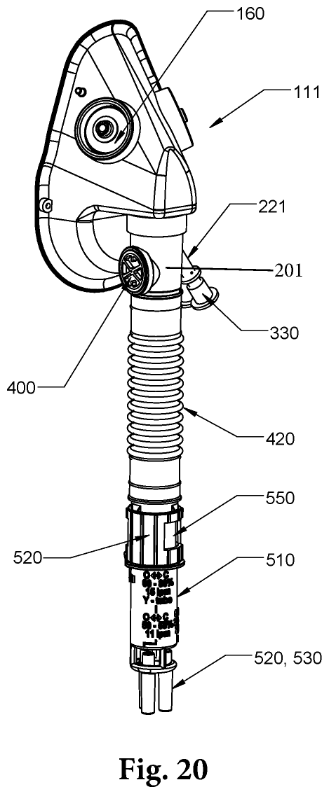

FIG. 20 shows yet another system which is an all in one venturi style oxygen delivery. It will be appreciated that this system uses the mask 111 of FIG. 14 as opposed to the mask 100 shown in other figures. An open distal end of the conduit member 201 is connected to corrugated tubing 420. The corrugated tubing 420 is also attached to venturi 500.

It will be appreciated that the wye connector 600 from FIG. 15 can be used in the same manner with the system of FIG. 20 in that the first branch 620 is attached to the oxygen side port 221 and the second branch 630 is attached to the venturi 500. The third leg of the wye connector 600 is connected to gas source 11. This arrangement provides high efficiency oxygen delivery.

It will be appreciated that it in this operation mode, there is no main inhalation valve.

The arrangement is FIG. 20 has the following features: 100% high precision aerosol/venturi delivery; 24%-85% all in 1 oxygen delivery venturi; and 24%, 28%, 35%, 40%, 50%, 55%, 60% and 80% oxygen concentrations.

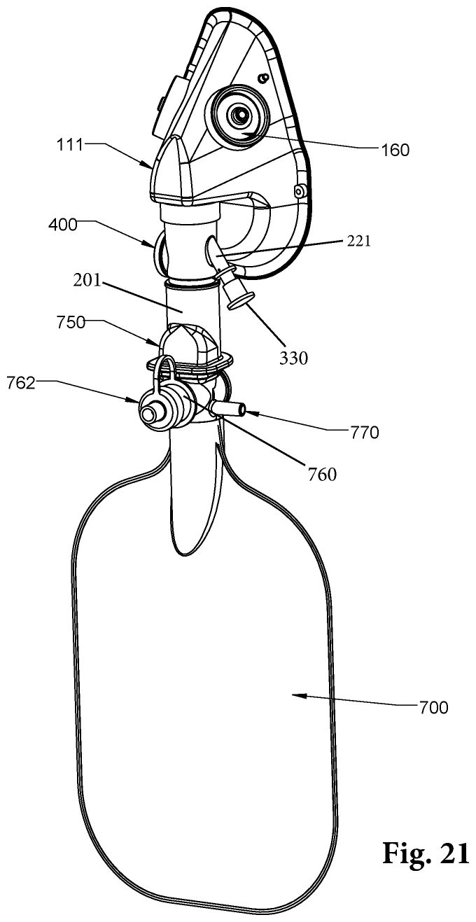

FIG. 21 shows a 100% nonrebreather/aerosol drug delivery system in an oxygen delivery mode. The system is formed of mask 111 with includes the integral conduit member 201. Attached to an open distal end of the conduit member 201 is the valve body connector 750 and therefore, the main inhalation valve (flapper 260) located inside the connector 750 is positioned upstream of the conduit 201 including the emergency inhalation valve 400.

In this operation mode, the front main port 760 is capped with cap (plug) 762. Supplemental gas (oxygen) can flow into the side port 770 and can flow directly into the bag 700 which is located upstream of valve 260. Thus, the oxygen can enter bag 700 along an unobstructed flow path (i.e., not having to flow through the valve 260 in the upper region of the connector 750).

Side port 221 is capped with cap 330.

The arrangement is FIG. 21 has the following features: 100% nonrebreather oxygen delivery; standard aerosol drug delivery; simultaneous oxygen and drug delivery (although only oxygen delivery is shown); and a supplemental air valve.

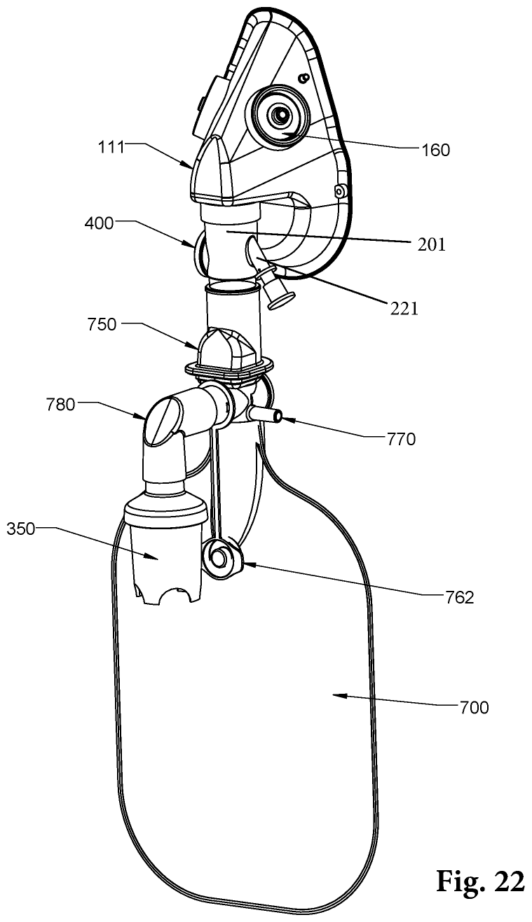

FIG. 22 shows a 100% nonrebreather/aerosol drug delivery system in drug delivery mode. The system is formed of mask 111 with includes the integral conduit member 201. Attached to an open distal end of the conduit member 201 is the valve body connector 750 and therefore, the main inhalation valve (flapper 260) located inside the connector 750 is positioned upstream of the conduit 201 including the emergency inhalation valve 400.

In this operation mode, the front main port 760 is fitted to elbow connector 780 which in turn is connected to nebulizer 350. Supplemental gas (oxygen) can flow into the side port 770 and can flow directly into the bag 700 which is located upstream of valve 260. Thus, the oxygen can enter bag 700 along an unobstructed flow path (i.e., not having to flow through the valve 260 in the upper region of the connector 750).

Side port 221 is capped.

The arrangement is FIG. 22 has the following features: 100% nonrebreather oxygen delivery; standard aerosol drug delivery; simultaneous oxygen and drug delivery; and a supplemental air valve.

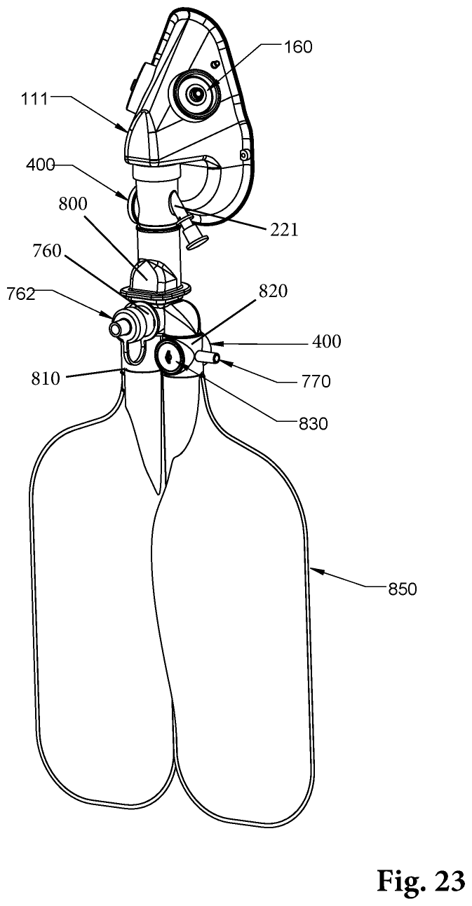

FIG. 23 shows a system in which the conduit member 201 is attached to the valve body connector 800. The side port 221 is capped and the front main port 760 is capped with cap 762. As in FIG. 19, the legs 810, 820 are attached to connectors/ports of the dual reservoir bag 850. Port 770 can be connected to a gas (oxygen) source and this gas freely flows into the second leg 820 and into the second compartment of the bag 850. Since both compartments of the bag 850 are upstream of the main inhalation valve, the oxygen from port 770 can flow into the compartments.

The arrangement is FIG. 23 can be thought of as being 100% nonrebreather/high efficiency aerosol drug delivery system (oxygen delivery mode) has the following features: 100% nonrebreather oxygen delivery; high efficiency drug delivery; simultaneous oxygen and drug delivery; and a supplemental air valve (emergency inhalation valve).

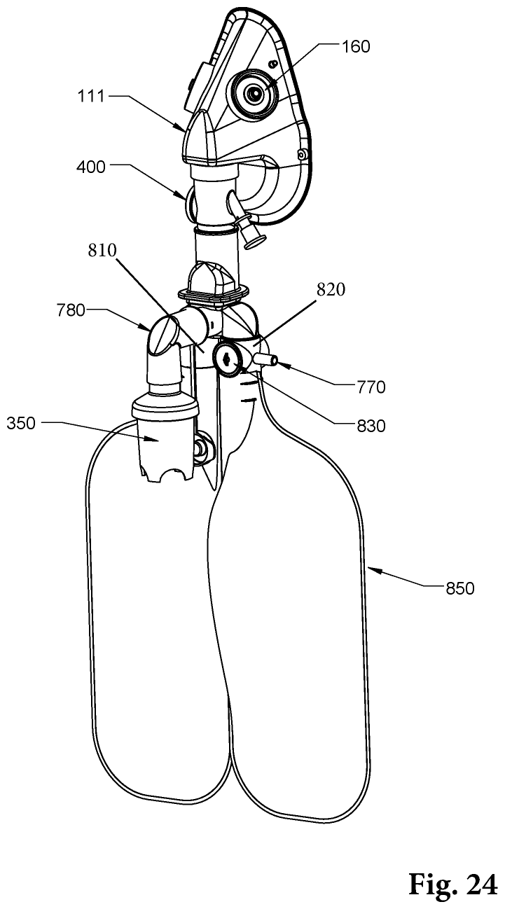

FIG. 24 shows a system in which the conduit member 201 is attached to the valve body connector 800. The side port 221 is capped and the front main port 760 is connected to elbow connector 780 which itself is connected to nebulizer 350. As in FIG. 19, the legs 810, 820 are attached to connectors/ports of the dual reservoir bag 850. Port 770 can be connected to a gas (oxygen) source and this gas freely flows into the second leg 820 and into the second compartment of the bag 850. Since both compartments of the bag 850 are upstream of the main inhalation valve, the oxygen from port 770 can flow into the compartments.

The arrangement is FIG. 24 which can be thought of as being a 100% nonrebreather/high efficiency aerosol drug delivery system (oxygen delivery mode) has the following features: 100% nonrebreather oxygen delivery; high efficiency drug delivery; simultaneous oxygen and drug delivery; and a supplemental air valve (emergency inhalation valve).

The modular pulmonary treatment system of the present invention provides a number of features and advantages not found in previous systems. These features and/or advantages include but are not limited to: (a) the use of a variable venturi 500 in conjunction with the disclosed mask 100 (patient interface device) with its integral oxygen port (port 220) allows for the delivery of a wider and more precise range of oxygen concentrations that what is commercially available; (b) by attaching a detachable swing valve 260 to the mask 100 with its integral oxygen port (port 220) in conjunction with a single reservoir bag and a wye split tubing set 600, allows for the delivery of 100% oxygen and 2× (two times) the standard dose of aerosolized medication; (c) by attaching a detachable swing or flapper valve 260 to the mask 100 with its integral oxygen port (port 220) in conjunction with corrugated tubing 370 having a valve assembly 400 at its distal end and a wye split tubing set 600 allows for the delivery of 100% oxygen and 1.5× the standard dose of aerosol medication; and (d) use of the mask 100 by itself or in conjunction with the detachable swing valve mechanism allows for attachment of multiple accessory devices thereby providing multiple respiratory treatments in a single system.

What is claimed is:

1. A modular pulmonary treatment system comprising:
a patient interface device defined by a body which defines a hollow interior;
a first inhalation valve associated with the body and located along an inhalation flow path for providing selective fluid communication with the hollow interior of the body; and
at least one exhalation valve assembly detachable coupled to the body for discharging exhaled gas from the hollow interior to atmosphere; wherein the at least one exhalation valve assembly comprises an exhalation valve cartridge including a housing having a perforated bottom wall with a post extending outwardly therefrom;
a valve member configured to seat against the perforated bottom wall and including an opening through which the post extends; and a valve retainer for placement over the post, whereby the valve member is securely held in place against the perforated bottom wall;
wherein the body includes at least one exhalation opening with the exhalation valve cartridge being secured to the body such that the exhalation valve cartridge covers the exhalation opening and the exhalation valve cartridge is detachable as a whole unit from the body.

2. The modular pulmonary treatment system of claim 1, wherein the patient interface device comprises a face mask.

3. The modular pulmonary treatment system of claim 1, wherein the housing further includes a perimeter wall that is upstanding relative to the perforated bottom wall and the body includes an upstanding wall that surrounds the exhalation opening with a ledge being defined between the upstanding wall and the exhalation opening, wherein the cartridge is disposed internally within the upstanding wall and seats against the ledge.

4. The modular pulmonary treatment system of claim 3, wherein a mechanical coupling is formed between the upstanding wall and the perimeter wall of the cartridge which are in intimate contact with one another.

5. The modular pulmonary treatment system of claim 3, wherein the perforated bottom wall seats against the ledge.

6. The modular pulmonary treatment system of claim 3, wherein the cartridge has a circular shape and the upstanding wall and exhalation opening are also circular in shape.

7. The modular pulmonary treatment system of claim 4, wherein the mechanical coupling is one of a frictional fit and a snap-fit.

8. The modular pulmonary treatment system of claim 1, further including a first conduit member that is detachably attached to an inlet port formed in the body, wherein the first inhalation valve is contained within the first conduit member.

9. The modular pulmonary treatment system of claim 8, wherein the first conduit member has a first end that has a first coupling structure and an opposite second end, wherein a second coupling structure surrounds the inlet port, the first and second coupling structure being complementary to result in a secure attachment of the first conduit member to the body.

10. The modular pulmonary treatment system of claim 9, wherein the first and second coupling structure form a snap-fit attachment.

11. The modular pulmonary treatment system of claim 8, wherein between the first and second ends, first and second side ports are formed in spaced relation to one another.

12. The modular pulmonary treatment system of claim 11, wherein the first side port comprises an elongated tubular structure that has an open distal end formed along one face of the first conduit member and the second side port is formed along another face of the first conduit member.

13. The modular pulmonary treatment system of claim 11, wherein the second side port includes an inhalation valve assembly.

14. The modular pulmonary treatment system of claim 11, wherein a detachable cap member is attached to the first side port, the detachable cap member includes a nipple and a plug that is on a tether and is configured to fit over and attach to the nipple.

15. The modular pulmonary treatment system of claim 9, further including: (a) a bellows type connector that is attached at a first end to the second end of the conduit member; (b) a second connector that is attached to a second end of the bellows type connector, the second connector having a side port in which an inhalation valve assembly is disposed; and (c) a source of aerosolized medication fluidly connected to the second connector.

16. The modular pulmonary treatment system of claim 9, further including: (a) a bellows type connector that is attached at a first end to the second end of the conduit member and (b) a venturi member attached to the bellows type connector and configured to be connected to a source of aerosolized medication.

17. The modular pulmonary treatment system of claim 16, further including a wye connector having a first branch connected to the venturi member and a second branch connected to the conduit member, the wye connector being fluidly connected to the source of aerosolized medication.

18. The modular pulmonary treatment system of claim 8, further including first and second conduit members, the first conduit member having a tubular main body and a side port extending outwardly therefrom for connection to a supplemental gas source, the first conduit member further including an emergency inhalation valve located along the tubular main body, the first conduit member being connected at a distal end to the second conduit member which includes the first inhalation valve.

19. A modular pulmonary treatment system comprising:
a patient interface device defined by a body which defines a hollow interior and includes a first connector extending outwardly therefrom;
at least one exhalation valve assembly coupled to the body for discharging exhaled gas from the hollow interior to atmosphere;
a valve body connector attached to the first connector, wherein the valve body connector includes an inhalation valve disposed therein; a main port for connection to a source of gas or a source of aerosolized medication; a supplemental gas port and an emergency inhalation valve, wherein the main port, the supplemental gas port and the emergency inhalation valve are all located upstream of the inhalation valve relative to the body of the patient interface device.
